(19) United States
(12) Patent Application Publication
(10) Pub. No.: US 2012/0232454 A1
(43) Pub. Date: Sep. 13, 2012

(54) Podiatric Applicator Device and Method

(75) Inventors: Marshall Cooper, Short Hills, NJ (US); Suzan F. Campbell, Ridgewood, NJ (US)

(73) Assignee: Westfield Foot and Ankle Specialists, LLC, Westfield, NJ (US)

(21) Appl. No.: 13/044,893

(22) Filed: Mar. 10, 2011

(51) Int. Cl. A61F 5/00

(52) U.S. Cl. 602/30

(57) ABSTRACT

A device and method to enable a user to clean or apply medicament to a foot that includes an elongated strip of pliant material adapted for insertion between toes and gripping by a user at respective ends of said strip, wherein the strip sufficiently thin to facilitate manipulation in a flossing motion between the toes.

### FIELD OF THE INVENTION

The present invention relates to the field of podiatric medical devices and particularly directed to devices and methods for cleaning and applying medicaments to the foot.

### BACKGROUND OF THE INVENTION

Treatment of foot disorders caused by injury or infection commonly requires cleaning and application of medicine to prevent bacterial or fungal infection or minimize further infection of the injured area. Even where physical injury is absent, common infections of the foot typically caused by fungus require treatment. Fungal infections are often the result of the environment or weakened immunity from disorders such as diabetes. This is especially true of the feet as they are often covered by socks and footwear creating a warm, dark, and humid environment encouraging fungus growth.

These fungal infections can irritate the skin on and between the toes. Symptoms include drying skin and itching. The affected area becomes inflamed and the skin in the region peels away by scaling, flaking, or blistering, and ultimately leading to exposed raw tissue that is reddish appearance and very sensitive to the touch with pain and swelling. Fungus will grow on and between toes as well as on soles and toenails. In addition, a localized infection will spread to toenails, the soles of the feet, and other parts of the body. Therefore timely treatment is important.

Prevention will minimize the possibility of such infections. Personal hygiene is essential to manage these fungal infections. Cleaning and drying the feet, especially between the toes, regularly can prevent the fungus that causes such infections from multiplying. In conjunction with regular good hygiene, conventional treatment typically involves daily or twice daily application of a topical medicament. The medicaments are usually in the form of a gel, cream, or ointment available either over-the-counter or by prescription. The medications are absorbed into the skin and kill the fungus and fungal root.

Treatment with topical medications requires applying the product directly to the affected skin of the feet and both on and in the interdiginous areas between toes. These interdiginous areas, particularly at the web of the toes, are difficult to reach. Further, those afflicted who have difficultly bending over due to weight, age, back problems, or other circumstances find hygiene and treatment of these areas difficult, if not impossible to do without assistance. Thus, application by hand may be painful and unsanitary. Moreover, a person may have difficulty bending or reaching their feet making application a challenge or impossible. Therefore, due to these difficulties treatment is made infrequently or avoided entirely.

Prior art devices, such as that described in U.S. Pat. No. 4,961,773, rely on a swab attached to a handle and permit a more sanitary application of medicaments. However, such devices lack the delicate touch and make uniformity of application difficult. Moreover, other prior art devices proposed for cleaning or application of chemicals to the feet are typically comprised of rigid handles with clumsy pads. Examples of such prior art devices include those described in U.S. Pat. No. 5,970,565. However, these devices lack the necessary flexibility to respond to contours of the toes to provide uniform product coverage. Also, such prior art devices fail to facilitate both delicate cleaning and application to the very sensitive skin.

### SUMMARY OF THE INVENTION

The disadvantages and inconveniences of the prior art devices are overcome by the present invention. The present invention is directed to a device and a method for treating a foot employing an elongated strip of fabric material that is sufficiently pliant to facilitate manipulation by a flossing motion in the interdiginous areas between toes. Such fabric strip is composed of a material for holding and applying a cleaning solution or medicament to the subject area of the foot. Correspondingly, a representative method in accordance with the invention comprises applying or impregnating a length of the sanitized fabric strip with the cleaning solution or medicament (or using a strip with the medicament pre-applied); grasping respective end portions by hand and manipulating the strip in a buffing or flossing motion over the foot region in need of treatment.

The present invention advantageously enables a user with her/his foot hygiene and application of medicaments, especially between the toes, with little effort and without requiring excessive bending. This provides a significant benefit for anyone with the task of cleaning and applying medicine to the toes, in particular to those individuals who are limited physically. The use of the flexible fabric enables the fabric to respond to the contours of the toes and enables more complete and uniform coverage over the affected areas in a far improved manner over prior art ridged handle and pad devices.

### BRIEF DESCRIPTION OF THE DRAWINGS

FIG. 1 is a diagram of an exemplary elongated flexible strip for cleaning and applying medicaments in accordance with the invention;

FIG. 2 is a diagram of an exemplary podiatric applicator dispenser useable with the present invention;

FIG. 3 is a cross-sectional cut-away view of the dispenser of FIG. 2; and

FIG. 4 illustrates an exemplary method of use of the podiatric applicator.

### DETAILED DESCRIPTION

Infection or injury of the foot is typically treated through a combination of thorough cleaning and treatment by the application of medicament, e.g., medicated ointment or powder, to the feet, toes, and the areas between the toes. Conventionally, one accomplishes this process by hand using either their fingers or a swab. The medications include, but are not limited to powders, creams and ointments, both over-the-counter and by prescription, and homeopathic remedies, such as tea tree ointment.

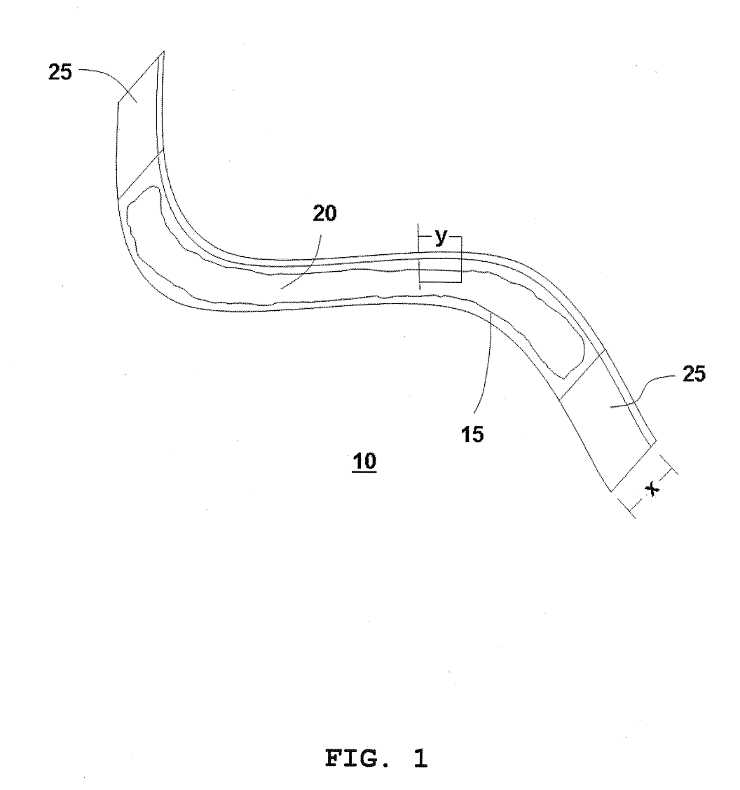

FIG. 1 illustrates an exemplary embodiment of the podiatric applicator in accordance with the present invention. The device consists of an elongated strip 10 of pliant material. The length of strip 10 is sufficiently long to at least facilitate a single treatment as described below. For example, a length in the exemplary range of 12 to 48 inches long would enable users to comfortably contact and manipulate the strip 10 within the interdiginous areas between toes without significantly bending or contorting their body. However, it is possible to greater or shorter lengths of strip 10 in accordance with the invention.

In FIG. 1, the thickness y of the strip 10 is sufficiently thin enable the strip to slide between toes without difficulty. Suitable thicknesses y for the strip 10 may be on the order of, for example, less than 0.25 inches. Correspondingly, FIG. 1 depicts the strip to having a width x. Suitable widths x for use with the strip 10 may be on the order, for example, of 0.20 to 3.0 inches.

The strip 10 is comprised of sufficiently pliant material to enable the strip 10 to be easily maneuvered by the user in a flossing or buffing motion in the interdiginous areas between toes and to be bent and twisted to conform to the skin in these areas. The strip 10 may be made of a variety of different materials. Depending upon the user's needs and comfort, materials may be selected based on varied absorbency, sterility, softness, or roughness. Suitable materials for strip 10 may be, for example, gauze, cotton, or synthetic materials. It may also be advantageous to utilize a sterilized material for strip 10. In an alternative embodiment, the strip 10 is scented to mask any unpleasant odor of the medicament or cleaner 20.

In those applications in which the strip 10 may be used for application of a medicament such as, for example, tea tree oil, anti-fungal powder, or talcum powder. It may be advantageous to pre-apply or impregnate the central region of the strip 15 with such medicament. FIG. 1 depicts a medicament 20 held by the material 15. Strip 10 has been sufficiently impregnated with medicament 20 to adequately treat the skin in the interdiginous areas between toes. In an alternative embodiment, a cleaning agent is held by the material 15 to clean the skin and areas between the toes. In a further alternative embodiment, the strip 10 is scented to mask any unpleasant odor of the medicament or cleaner 20.

It is advantageous to avoid providing the medicament 20 to respective first and second end portions 25 of the strip 10 that are intended to be gripped by the user. The purpose of leaving the respective end portions 25 free of medicament 20 is to allow a person to grasp the strip 10 without exposing their hands to the medicament. In alternative embodiments of the invention, the two respective end portions 25 are colored, shaded or textured differently for ease of identification and or gripping.

It is also possible to practice the present invention without pre-treating strip 10. In such manner, the strip 10 may be used for cleaning or a user may manually apply a cleaner or medicament to the non-pretreated strip 10. The strip 10 may be comprised of a single ply or multi-ply material. In another embodiment, the strip 10 may be comprised of folded layers or of rolled form. In yet another embodiment a plurality of strips 10 are connected to one another for dispensing, wherein the first end portion of a first strip is connected to a second end portion of a second strip by perforations.

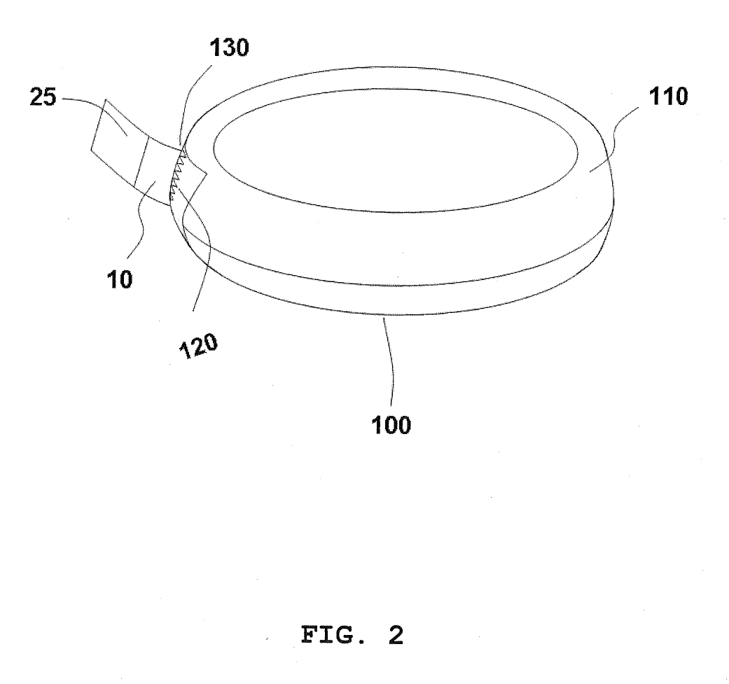

For further ease of use, a plurality of strips 10 of predetermined lengths may be conveniently contained in a dispenser. For example, a plurality of strips 10 may be connected to one another, wherein the first end portion of a first strip is connected to a second end portion of a second strip by perforations. In the alternative, the length of strip 10 is not pre-determined and the user obtains an appropriate length for her/his individual needs from an extended length of pliant material arranged in, for example, a reel and packaged in the exemplary dispenser 100 depicted in FIG. 2. In FIG. 2, the strip 10 is dispensed by the dispenser 100 through a slot-like opening 120 of housing 110. FIG. 2 further illustrates a cutter 130 proximate opening 100. The cutter 130 includes, for example, a metal serrated edge, for cutting the strip 10 for dispensing.

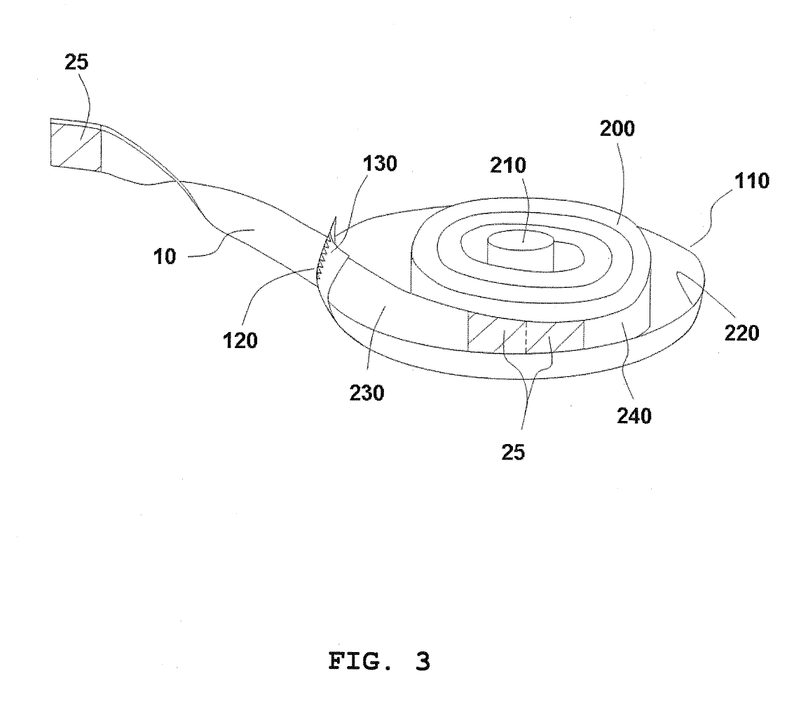

FIG. 3 depicts a cross-sectional cut-away view of the dispenser 100 of FIG. 2. In FIG. 3, a plurality of strips 10 (shown in FIG. 1) are rolled along their length, forming a reel 200. The reel 200 is rotatably connected by a spindle 210 to the inside wall 220 with in the cavity of dispenser housing 110. FIG. 3 further shows the end portions 25 as textured of adjacent strips 10, as identified by reference numbers 230 and 240.

It is possible to employ a dispenser housing that omits the cutter 130. For such an emobodiment, it is advantageous for the reel 200 to include perforations between adjacent strips 10 allowing a user to dispense a strip 10 by tearing at the perforation. It is also possible to utilize a dispenser housing or packaging with the present invention that maintains a seal with respect to dispensing strips 10 to preserve and protect any medicament maintained in the dispenser or impregnated in the strips 10. Alternatively, the present invention may rely on a dispenser (not shown) for holding separate, individual strips 10 (shown in FIG. 1) that are folded and arranged in such a manner to be dispensed one at a time in a pop-up manner or otherwise.

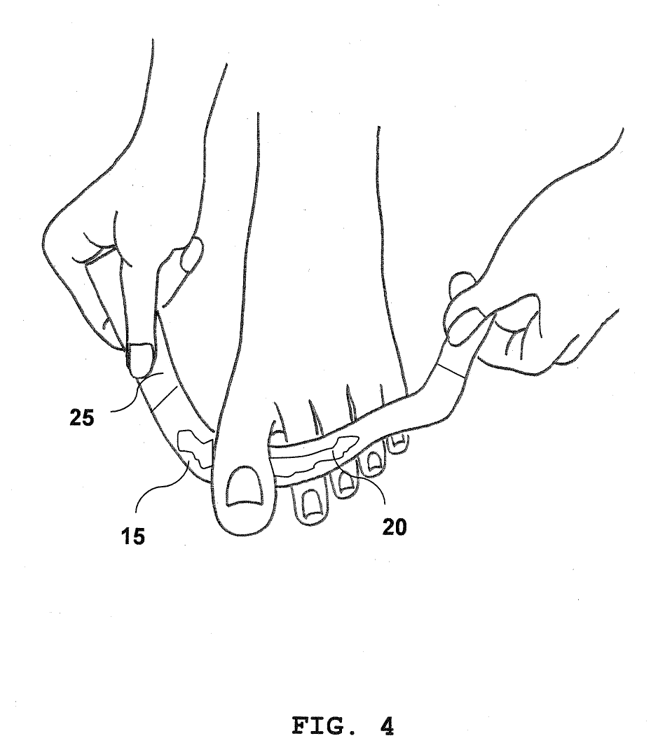

A method of applying medication using the podiatric applicator is described with reference to the foot illustration of FIG. 4. As shown in FIG. 4, a user may grasp the first and second end portions 25 of the strip 10. The strip 10 in FIG. 4 includes the medicament 20 in a central region 15 and not at the respective ends. The user then manipulates the strip 10 between around the skin of the toes, the web areas between the toes using a flossing or buffing motion ensuring coverage and transfer of medicament to the affected area.

In an alternative the user may take a sufficient length of a non-medicated strip, apply medicament or cleaner to the strip, and maneuver the strip around the skin of the affected areas in a manner substantially identical to that depicted in FIG. 4. In yet another embodiment, the user will maneuver a length of the non-medicated strip to ensure the skin is suitably dry. It is contemplated that due to the ease of use of this device and method anyone may use this invention including: patients, assistants, a medical providers, and medical specialists.

It should be understood that while the present invention has been described with respect to disclosed embodiments, numerous variations are possible without departing from the spirit and scope of the present invention as defined in the claims. For example, the present invention may implemented with strips having central regions having medicaments or cleaners disposed thereon and end portions comprised of different materials. Moreover, it is intended that the scope of the present invention include all foreseeable equivalents to the elements and structures as described herein and with reference to the drawing figures. Accordingly, the invention is to be limited only by the scope of the claims and their equivalents.

What is claimed is:

1. A podiatric applicator device comprising:
an elongated strip of pliant material adapted for insertion between toes and gripping by a user at respective ends of said strip; and
the strip having a thickness capable of manipulation in a flossing motion between the toes.

2. The device of claim 1, further comprising:
a medicament or cleaning agent disposed on a central region of the strip.

3. The device of claim 2, wherein at least a region of said strip is impregnated with medicament or cleaning agent.

4. The device of claim 2, wherein said strip is of a predetermined length adapted for a single treatment and the end portions are indicated by a color different from the medicated portion.

5. The device of claim 2, wherein said strip is of a predetermined length adapted for a single treatment and the respective end portions are the length of the first and second end portions are textured differently from the medicated portion.

6. The device of claim 2, wherein the strip is scented.

7. The device of claim 1, wherein the strip is made of cotton.

8. The device of claim 1, further comprising:
a plurality of said strips connected to one another wherein the first end portion of a first strip is connected to a second end portion of a second strip.

9. The device of claim 8, wherein said connections between strips are perforated connections.

10. The device of claim 8, wherein said plurality of strips are rolled and arranged in a reel for dispensing.

11. The device of claim 8, further comprising:
a housing defining a cavity therein;
said rolled strips being rotatably mounted to the housing within said cavity; and
wherein said housing includes an opening for dispensing the strips.

12. The device of claim 11, further comprising:
a cutter disposed proximate said housing opening for cutting said strips.

13. The device of claim 8, wherein said material is a multi-ply material.

14. The device of claim 8, wherein said material is a folded material.

15. The device of claim 8, wherein said material is a rolled material along its length.

16. A method of applying medicine to toes using an elongated strip of pliant material having a thickness capable of manipulation in a flossing motion between the toes, said method comprising the steps of:
grasping by hand the respective end portions of the strip with cleaning agent or medicament impregnated into or disposed of onto the strip;
contacting the strip to the area in need of treatment; and
manipulating the strip around the area in need of treatment.

17. A method of manufacture of a podiatric applicator device, comprising the steps of:
rolling a plurality of the elongated strips connected to one another wherein the first end portion of a first strip is connected to a second end portion of a second strip along its length; and
rotatably connecting the rolled material to the housing
disposing a cutter at an opening of said housing adapted for cutting said material; and
dispensing the strip through the opening.
